(19) United States
(12) Patent Application Publication
(10) Pub. No.: US 2006/0258980 A1
(43) Pub. Date: Nov. 16, 2006

(54) Global myocardial perfusion catheter

(75) Inventors: Charles R. Bridges, Villanova, PA (US); Kapil Gopal, Wallingford, PA (US)

Correspondence Address: HOWSON AND HOWSON, SUITE 210, 501 OFFICE CENTER DRIVE, FT WASHINGTON, PA, 19034, US

(73) Assignee: The Trustees of the University of Pennsylvania, Philadelphia, PA (US)

(21) Appl. No.: 10/572,238

(22) PCT Filed: Sep. 16, 2004
(86) PCT No.: PCT/US04/30463

§ 371 Date: May 30, 2006

Related U.S. Application Data
(60) Provisional application No. 60/504,743, filed on Sep. 19, 2003.

(51) Int. Cl. A61M 29/00

(52) U.S. Cl. 604/101.05

(57) ABSTRACT

A catheter (40, 40A, 40B) and method for retrograde perfusion of the heart through the coronary sinus is provided. In one embodiment, the catheter (40, 40A, 40B) has a pair of separate balloons (66, 66A, 66B, 68, 68A, 68B) that are located side-by-side on its distal end (44, 44A, 44B) and that can be positioned and expanded on opposite sides of the coronary sinus ostium to anchor the catheter to the coronary sinus and to form an occlusive seal of the coronary sinus adjacent its ostium. Alternatively, a single asymmetric “dumbbell” or “hourglass” shaped balloon can be used such that one portion of the balloon inflates in the right atrium, and thereafter, the other portion of the balloon is inflated at a location crossing the ostium and extends into the coronary sinus to occlude the ostium through radial expansion against the sinus at the ostium. The catheter and its method of use provide a tighter seal and improve distribution of cardioplegia and/or other substances, such as drugs or gene therapy vectors, to the myocardium.

### BACKGROUND OF THE INVENTION

The present invention relates to a catheter for retrograde perfusion of the heart through the coronary sinus, and more particularly, to a balloon catheter that provides improved distribution of cardioplegia or other substances, such as drugs or gene therapy vectors, to the myocardium.

During surgery involving cardioplegic arrest with cardiopulmonary bypass, the myocardium requires a constant supply of cardioplegia to maintain coronary perfusion and prevent intra-operative myocardial damage. Cardioplegia can be delivered antegrade, through the coronary ostia, or retrograde, via the coronary sinus. Retrograde cardioplegia delivery is preferred in certain situations, such as, with patients having significant aortic insufficiency, or in patients with diffuse coronary arterial disease. The minimal number of valves within coronary veins and the extensive degree of collateralization between the coronary artery and veins enable the use of retrograde coronary sinus perfusion.

Examples of retrograde perfusion coronary sinus balloon catheters and of techniques for retrograde cardioplegia delivery via the coronary sinus are described in U.S. Pat. No.: 4,927,412 issued to Menasche; U.S. Pat. No. 5,021,045 issued to Buckberg et al.; U.S. Pat. No. 5,385,548 issued to Williams et al.; U.S. Pat. No. 5,707,358 issued to Wright; U.S. Pat. No. 5,779,685 issued to Thompson et al.; U.S. Pat. No. 6,500,145 B1 issued to Bicakci et al.; U.S. Pat. Nos. 5,620,418 and 5,807,326 issued to O'Neill et al.; and U.S. Pat. Nos. 5,913,842, 5,738,652 and 5,558,644 issued to Boyd et al.

According to conventional practices as illustrated in FIG. 1, a retrograde perfusion catheter 10 is inserted into the coronary sinus 20 and utilizes an inflatable balloon 12 to prevent dislodgement of the catheter 10 from the coronary sinus 20 and to form an occlusive plug within the coronary sinus 20. Typically, the tip 14 of the distal end 18 of the catheter 10 is inserted several centimeters into the coronary sinus 20 a significant spaced-distance “D” inward of the coronary sinus ostium (ie., orifice or mouth) 22 that is formed in a wall 24 of the right atrium 26. This spacing “D” ensures that the catheter 10 does not slip out of the coronary sinus 20 into the right atrium 26 of the heart, for instance, during a surgical procedure requiring multiple manipulations of the heart Inadequate myocardial protection and myocardial damage can occur if the catheter slips out of the coronary sinus without the surgeon's knowledge.

At least two problems are created with the catheter placement discussed above. One problem relates to the inability to provide a direct path of delivery of a solution or substance to the right ventricle (see the schematic view of FIG. 1) and the other relates to venous “shunting” or “steal” of the delivered solution or substance into coronary veins which drain into more proximal portions of the coronary sinus relative to the catheter's balloon location within the coronary sinus (see the diagram of FIG. 2).

Catheter placement as illustrated in FIG. 1 may provide adequate delivery of cardioplegia to the left side of the heart (ie., the left ventricle and left atrium) via multiple large coronary veins that coalesce to form the great cardiac vein from the left ventricle and the oblique vein of the Marshall from the left atrium. See reference numeral 28 in FIG. 1. However, such placement does not provide direct cardioplegia to the right ventricle. The majority of circulation from the right ventricle into the coronary sinus is provided via the small cardiac vein and posterior interventriclular vein (ie., middle cardiac vein). See reference numeral 30 in FIG. 1. The small and middle cardiac veins 30 communicate with the coronary sinus 20 adjacent the coronary sinus ostium 22. Thus, since the balloon 12 of the catheter 10 is positioned a spaced distance “D” inward of the ostium 22, flow of cardioplegia directly into the right ventricle via the small and medium cardiac veins 30 is prevented.

The simplified diagram of FIG. 2 illustrates the problem relating to “shunt” or “steal” pathways. As before, reference numerals 20, 22, 24 and 26 identify the coronary sinus, the ostium, the wall of the right atrial chamber, and the right atrium, respectively, and reference numerals 32 and 34 identify cardiac muscle and coronary veins, respectively. As shown by the arrows in FIG. 2, the placement of the tip 14 and balloon 12 of the catheter 10 a required distance “D” from the ostium permits the shunting or steal of the delivered solution and/or substance via coronary veins which drain into more proximal portions of the coronary sinus adjacent a proximal side of the balloon 12. This permits an amount of the delivered solution/substance to leak back through the ostium into the right atrium and reduces the pressure gradient of the solution/substance within the heart.

There is need for a retrograde perfusion coronary sinus catheter and method capable of providing improved global delivery of cardioplegia, drugs, gene therapy vectors, or the like to the heart.

### BRIEF SUMMARY OF THE INVENTION

Advantageously, the present invention provides a catheter that meets these needs. This catheter provides maximal occlusion of the myocardial venous return system and is capable of being safely and reliably secured to the coronary sinus in a manner that protects the cardiac muscle from injury at the location of entry into the coronary sinus. In addition, such a catheter should provide a tight seal at the most proximal portion of the coronary sinus and should prevent unrestricted flow from large, anastamosing coronary veins back into the right atrium. Further, the catheter should enable direct delivery to the right ventricle of the heart via the small and middle cardiac veins at a maximal pressure gradient.

### BRIEF DESCRIPTION OF THE DRAWINGS

The foregoing and other objects, features and advantages of the present invention should become apparent from the following description when taken in conjunction with the accompanying drawings, in which:

FIG. 1 is schematic view of a prior art retrograde perfusion coronary sinus catheter and its conventional positioning within a coronary sinus according to the prior art;

FIG. 2 is simplified diagram of a prior art retrograde perfusion coronary sinus catheter and its conventional positioning within a coronary sinus according to the prior art;

FIG. 3 is a perspective view of a retrograde perfusion catheter according to the present invention;

FIG. 4 is a schematic view of a retrograde perfusion coronary sinus catheter according to the present invention properly positioning within a coronary sinus;

FIG. 5 is simplified diagram of a second embodiment of a retrograde perfusion coronary sinus catheter according to the present invention and its proper placement within a coronary sinus; and

FIG. 6 is simplified diagram of a third embodiment of a retrograde perfusion coronary sinus catheter according to the present invention and its proper placement within a coronary sinus.

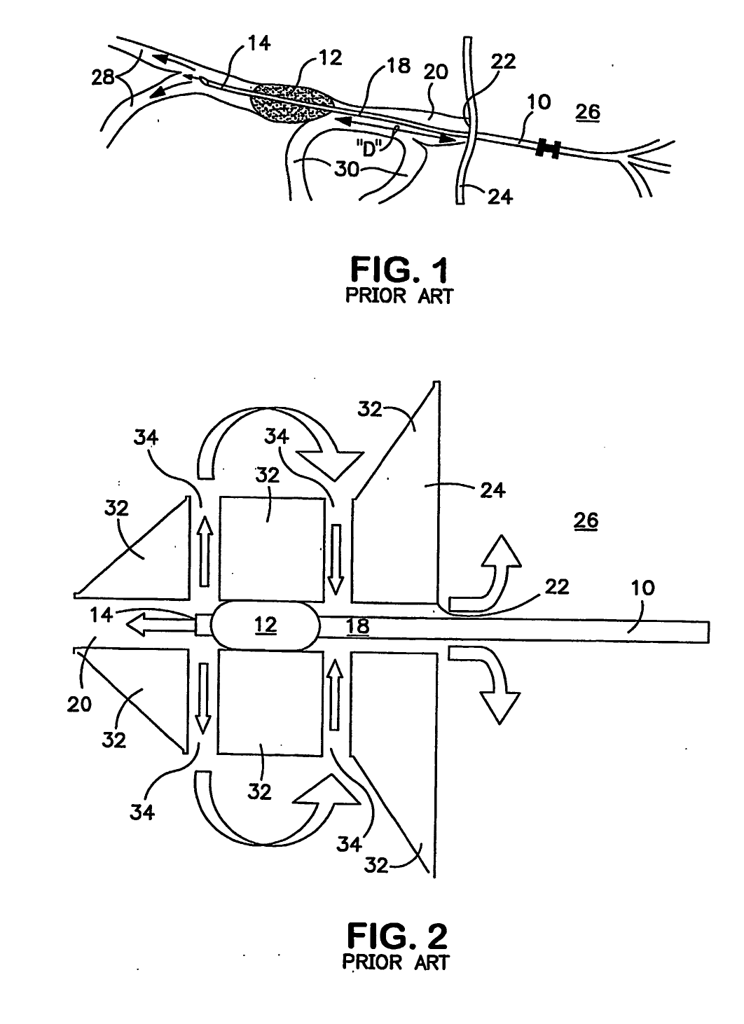

### DETAILED DESCRIPTION OF THE INVENTION

The present invention provides a retrograde perfusion catheter having a flexible, elongate cannula of a size capable of insertion into a coronary sinus of a heart. The cannula has a proximal end, a distal end, and an infusion lumen extending longitudinally s within the cannula with an outlet adjacent the distal end. A first inflatable balloon, or balloon portion, is attached to and surrounds a length of the cannula a spaced distance upstream of the infusion lumen outlet and is inflatable to a size greater than the size of a coronary sinus ostium. A second inflatable balloon, or balloon portion, is also attached to, and surrounds, a length of the distal end of the cannula adjacent to the first balloon, or balloon portion. At least a portion of the second balloon extends closer to the infusion lumen outlet than the first balloon. The second balloon is inflatable from a size capable of introduction into the coronary sinus to an inflated size for engagement with the walls of the coronary sinus. Positioning and inflation of at least portions of the first and second balloons on opposite sides of the coronary sinus ostium enables the catheter to be anchored to the coronary sinus and enables a substantially fluid-tight seal to be formed at the ostium.

According to another aspect of the present invention, a method for retrograde coronary sinus perfusion of a patient's heart with cardioplegia, drugs, gene therapy vectors, or other solutions or substances is provided. A catheter is provided having a flexible, elongate cannula with proximal and distal ends, an infusion lumen extending longitudinally within the cannula with an outlet adjacent the distal end, and a pair of adjacent balloon portions located on the distal end. The distal end of the cannula is inserted into the coronary sinus so that one of the balloon portions is positioned within the coronary sinus and the other of the balloon portions is positioned in the right atrium of the heart adjacent and exterior the coronary sinus. The balloon portions are expanded to anchor the catheter to the coronary sinus and to completely seal the coronary sinus from the right atrium. Thereafter, a solution/substance is injected into the infusion lumen so that the solution/substance flows through the outlet of the cannula into the coronary sinus and is prevented from flowing back through the ostium into the right atrium.

The catheter of the invention is thus useful for delivery of a macromolecular complex and permits delivery of such a complex under high hydrostatic pressure, while protecting the cardiac sinus.

As used herein, the term “macromolecular complex” encompasses any biologically useful moiety that can be transferred into targeted cells (e.g., muscle cells). Examples of suitable macromolecular complexes include vectors composed of nucleic acids, including DNA and RNA molecules, an enzyme, a protein, peptide, or non-proteinaceous molecule, which may include small molecules or other chemical moieties. The macromolecular complexes of the invention are not limited by size, but rather encompass molecules that, due to their large size, are not able to enter the cell on their own as well as molecules that can infect or transfect cells without the application of the present method. A vector includes plasmids, episomes, cosmids, viral vectors, phage, “naked DNA”, any of which desirably contains a transgene under the control of regulatory sequences that direct expression thereof in a target cell. In one embodiment, the macromolecular complex comprises a viral vector. Examples of suitable viral vectors include, without limitation, adenoviruses, picomavirus, adeno-associated viruses, retroviruses, baculoviruses, and lentiviruses, among others.

The transgene is a nucleic acid sequence, heterologous to the vector sequences flanking the transgene, which encodes a polypeptide, protein, or other product, of interest The nucleic acid coding sequence is operatively linked to regulatory components in a manner that permits transgene transcription, translation, and/or expression in a host cell.

As used herein, the term “high hydrostatic pressure” generally refers to a pressure in the range of 50 mm Hg to 500 mm Hg. Suitable pressures within this range, e.g., 75 mm Hg, 100 mm Hg, 150 mmHg, 200 mm Hg, 250 mm Hg, 300 mm Hg, 350 mm Hg, 400 mm Hg, or 450 mm Hg, or others within or outside this range may be readily selected. High hydrostatic pressure is applied according to the invention by a low resistance (large bore) catheter or cannula in either a vein or artery, or by other methods that will be readily apparent to one of skill in the art.

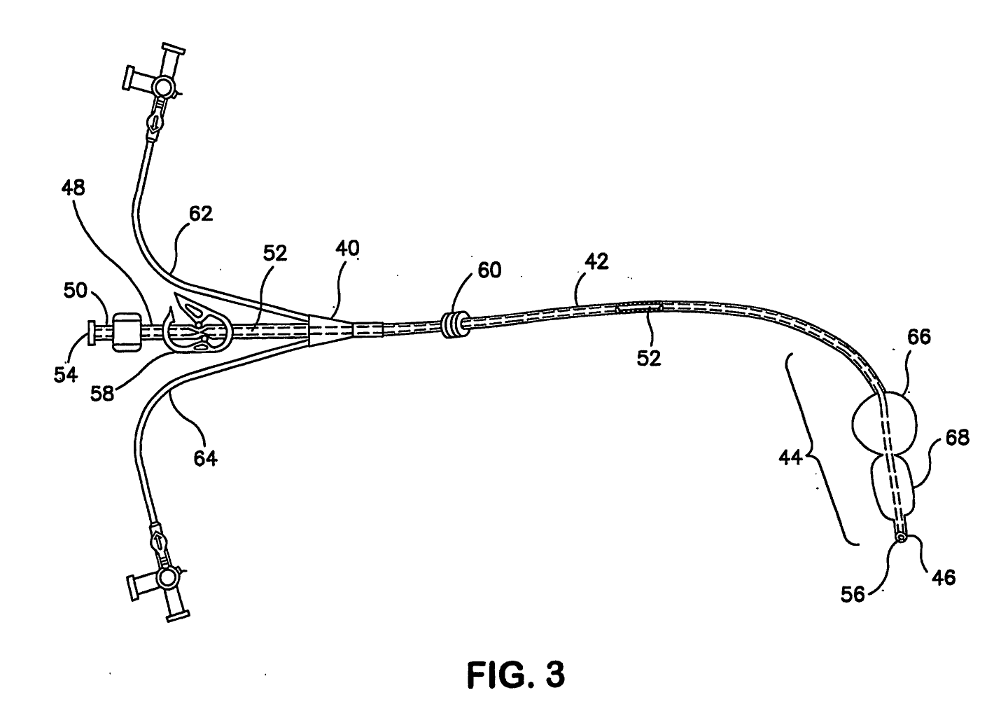

FIG. 3 illustrates a retrograde perfusion catheter 40 according to one embodiment of the present invention. The catheter 40 has an elongate, flexible cannula 42 of variable length, depending on its clinical use. The cannula 42 has a distal end 44 with a tip 46 and a proximal end 48 with a connector 50 for attaching the catheter 40 to a source of cardioplegic or other solution (not shown). At least the distal end 44 of the cannula 42 is of a size, or diameter, for introduction into the coronary sinus of the heart via the coronary sinus ostium which is formed by tissue of the wall of the right atrium. A movable hub 60 can be located on the catheter 40 for use in securing the catheter 40 outside of the heart, such as with sutures, during a surgical procedure to help minimize movement of the catheter 40 after the tip 46 is located within the coronary sinus.

At least one lumen, more preferably, multiple lumen extend longitudinally within the cannula 42 between its proximal and distal ends, 48 and 44. The primary and largest lumen is infusion lumen 52 which provides a path of flow for the cardioplegic or other solution The infusion lumen 52 has an inlet 54 at the proximal end 48 of the cannula 42 and an outlet 56 at the tip 46 of the distal end 44 of the cannula 42 so that the cardioplegic or other solution can flow the full length of the cannula 42 and exit through the tip 46 into the coronary sinus. Preferably, a clamp 58 or the like is located on the proximal end 48 of the cannula 42 to seal the infusion lumen 52 when desired.

Additional secondary lumen, 62 and 64, can also extend longitudinally within the cannula 42 between its proximal and distal ends, 48 and 44. For instance, the secondary lumen can be used to supply a fluid for expanding inflatable balloons, discussed in greater detail below, and/or can be utilized to monitor the pressure within the coronary sinus during a surgical procedure. Alternatively, or in addition thereto, a secondary lumen can be provided for the pressure transduction or delivery of a second substance, such as intracardiac macromolecular complexes, pharmaceutical agents, gene therapy products, and the like to the coronary sinus during a surgical procedure.

The catheter 40 illustrated in FIG. 3 includes a pair of separate inflatable, or expandable, balloons 66 and 68, or equivalent expandable bladder structures, extending about a length of the distal end 44 of the cannula 42. Preferably, each of the balloons, 66 and 68, is substantially annular and surrounds a length of the cannula 42 which extends therethrough.

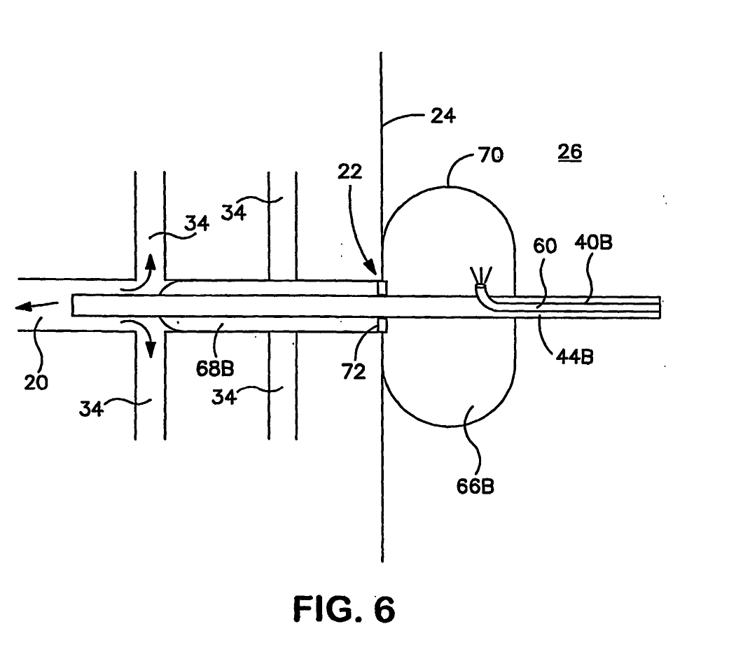

The first balloon 66, also referred to herein as the “atrial balloon”, is located on the cannula a spaced distance from the tip 46 of the cannula 42, and at least a portion of the second balloon 68, also referred to herein as the “distal balloon”, extends distally of the atrial balloon 66 closer to the tip 46 of the cannula. The embodiment of the present invention illustrated in FIG. 3 includes an atrial balloon 66 that is located adjacent the second balloon 68 in a side-by-side manner with little or no spacing therebetween. Other balloon configurations are also contemplated. For example, at least portions of the balloons can overlap as best illustrated in FIG. 5, or a single balloon can be provided having two differently sized inflatable sections as best illustrated in FIG. 6.

The second, or distal, balloon 68 should be of a size capable of insertion into the coronary sinus in a deflated condition and capable of expansion into engagement with the inner walls of the coronary sinus when inflated. Preferably, balloon 68 has a diameter that is generally the same size, or slightly larger than, the diameter of the distal end 44 of the cannula 42 when the balloon 68 is in a deflated condition. After inflation within the coronary sinus, balloon 68 should be of a size to form an occlusive plug within the coronary sinus adjacent the ostium and should prevent flow from the large, anastamosing coronary veins back into the right atrium through the ostium. Such a seal enables improved retrograde perfusion of the entire heart at enhanced pressure gradients and eliminates shunt, or steal, pathways.

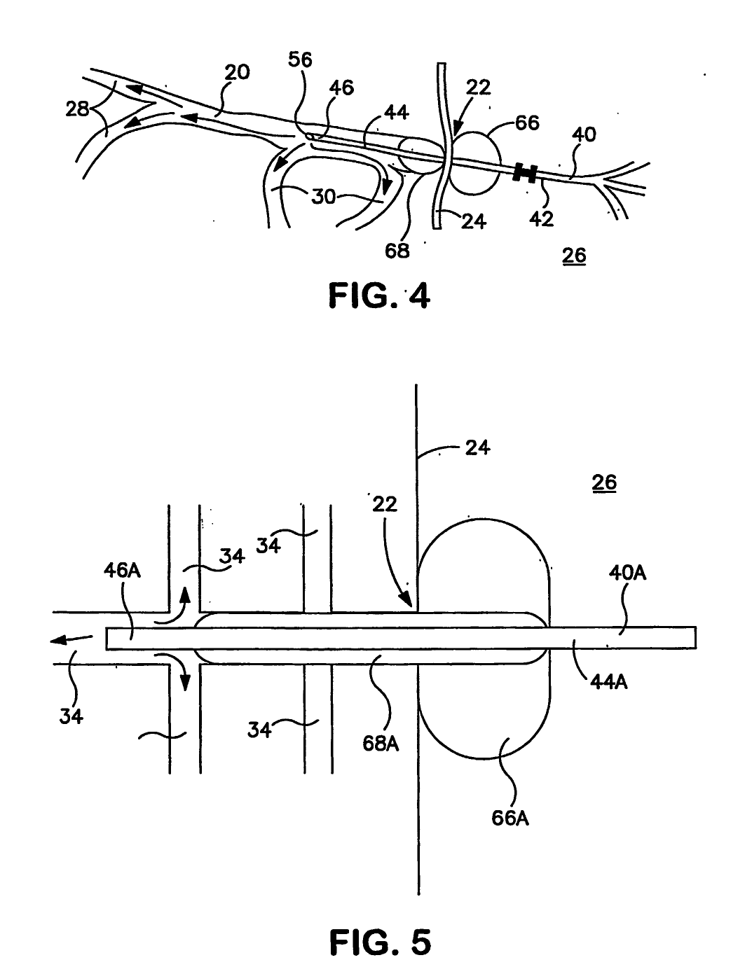

The first, or atrial, balloon 66 should be capable of expanding to a size, or diameter, greater than that of balloon 68 and greater than that of the coronary sinus ostium. Thus, when at least a portion of the distal balloon 68 is positioned and inflated within the coronary sinus and when the atrial balloon 66 is positioned and inflated at a location adjacent the ostium exterior of the coronary sinus in the right atrium, the catheter 40 is securely anchored to coronary sinus and should be prevented from becoming unintentionally dislodged from the coronary sinus. To this end, the ostium and the tissue of the wall of the right atrial chamber surrounding the ostium are sandwiched and captured between the inflated pair of balloons, 66 and 68. This serves to reliably anchor the catheter 40 to the coronary sinus at the ostium. In addition, as discussed above, the balloons 66 and 68 seal the ostium and prevent solution from passing out of the coronary sinus through the ostium into the right atrium. This is best illustrated schematically in FIG. 4.

An alternate embodiment of the configuration of the balloons is illustrated in the diagram of FIG. 5. The distal end 44A of catheter 40A has a pair of balloons, 66A and 68A, that partially overlap. The distal balloon 68A extends along a greater length of the catheter 40A than the atrial balloon 66A, and a portion of distal balloon 68A extends closer to the tip 46A. The atrial balloon 66A overlaps a portion of balloon 68A and extends, when inflated, to a greater diameter. See FIG. 5.

Another alternate embodiment of the configuration of the balloon of a catheter according to the present invention is illustrated in the diagram of FIG. 6. The distal end 44B of catheter 40B has a single asymmetric balloon 70 with opposite interconnected portions 66B and 68B intended to traverse the coronary sinus ostium when inflated. The single balloon 70 can have a generally “dumbbell” or “hour-glass” shape, for instance, as shown by the outline of the balloons in FIG. 3 or it can simply have different shaped sections as shown in FIG. 6. Due to its length and shape, the balloon 70 is prevented from becoming dislodged from the coronary sinus upon inflation with the balloon 70 crossing the ostium. For example, the balloon 70 can be positioned in a patient's heart such that one portion, 68B, inflates in the right atrium and the other portion, 66B, inflates, crosses the ostium, and extends into the coronary sinus thereby occluding the ostium through radial expansion against the coronary sinus at the ostium.

With respect to inflating and/or expanding the balloon or balloons of any of the above referenced embodiments, a pair of separate inflation lumens can be provided so that each balloon, or balloon portion, can be inflated/deflated separately of the other balloon or portion in sequence. For example, the atrial balloon 66 can be inflated in the right atrium, and thereafter, the tip of the cannula can be advanced into the coronary sinus until the atrial balloon 66 engages the ostium exterior the coronary sinus. Thereafter, the distal balloon 68 can be inflated within the coronary sinus to anchor the catheter and seal the ostium.

Alternatively, a single inflation lumen can extend through the cannula and communicate with both balloons, 66, 68, 66A and 68A, or balloon portions, 66B and 68B, for sequentially or simultaneously inflating or deflating the balloons or balloon portions. For example, the balloon 70 may have asymmetric and nonlinear stretch/strain capacitances or a threshold mechanism where only one lumen, such as lumen 60, is required to inflate both portions, 66B and 68B, but not necessarily simultaneously. For example, portion 66B in the right atrium may be inflated before the portion 68B is fully extended into and inflated within the coronary sinus. A diaphragm-type valve 72 or the like can extend between the balloon portions, 66B and 68B, to permit one balloon portion to substantially fully expand before the valve opens to permit flow into the other balloon portion. Alternatively, the stretch/strain nature of the balloon material can be such to permit one balloon to expand when a first inflation pressure is applied and the other balloon to expand only when a greater amount of inflation pressure is applied.

According to another alternative, the infusion lumen 52 can communicate with one or both of the balloons, or balloon portions, so that the balloons, or balloon portions, are automatically expanded when cardioplegic solution or the like is flowed through the catheter 40. In this case, the solution would first flow into one or both balloons, 66 and 68, to expand the balloons and then into the coronary sinus through the tip 46.

By way of example, and not be way of limitation, the cannula 42 can be made, for instance, of a flexible thermoplastic material, thermoplastic elastomer, thermoset elastomer, polyvinylchloride, polyurethane, polyethylene, polypropylene, polyamides, polyesters, silicone, latex, and alloys and copolymers thereof, as well as braided coiled or counterwound wire or filament reinforced composites. The distal balloon can be expanded, for instance, to an outer diameter of about 6 to 20 mm, and the atrial balloon can be expanded, for instance, to an outer diameter of about 10 to 30 mm. Of course, the balloons can be of other sizes, as needed, and both should be capable of fitting through a small incision prior to expansion. The balloons, 66 and 68, can be made of flexible polymers and elastomers including polyvinylchloride, polyurethane, polyethylene, polypropylene, polyamides, polyesters, silicone, latex, silicone, and alloys, copolymers and reinforced composites thereof. The balloons, 66 and 68, can be secured to the catheter utilizing various technologies including adhesive bonding, heat welding, wrapping with a winding filamentary material, or combinations thereof.

Use of the retrograde perfusion catheter according to the present invention can be applied to experimental and clinical medicine/science in animals and humans. The catheter is compatible with technologies already in use for open-heart surgery and can be used for the global delivery of any substance to the myocardium.

In use, the distal end of the dual-balloon catheter is advanced into the coronary sinus 20 via its ostium 22 utilizing any of the techniques described in the previously mentioned U.S. patents. However, according to the present invention the distal end is inserted such that only the tip 46 and distal balloon are located within the coronary sinus 20 adjacent the coronary sinus ostium 22 while the atrial balloon is positioned exterior of the coronary sinus 20 in the right atrium 26 adjacent the wall 24 of the right atrial chamber which surrounds and defines the ostium 22. Thereafter, both balloons, 66, 68, 66A and 68A, or balloon portions, 66B and 68B, are inflated thereby capturing the tissue of wall 24 therebetween to anchor the catheter to the coronary sinus 20 and to seal the coronary sinus 20 at it ostium 22. Preferably, the atrial balloon, 66, 66A, and 66B, is inflated to a larger diameter than the distal balloon, 68, 68A, and 68B.

Thereafter, a solution is flowed through the infusion lumen 52 and into the coronary sinus 20 downstream of the distal balloon 68, 68A, and 68B. Preferably, the solution is permitted to flow directly into the medium and small cardiac veins 30 via their junction with the coronary sinus 20. See FIG. 3. This permits the direct flow of solution at a desired pressure gradient to the right ventricle of the heart.

If desired, the method of using the catheter can include simultaneous inflation/expansion of the balloons 66, 68, 66A, 68B, or balloon portions, 66B and 68B. For instance, the solution being infused into the coronary sinus can be directed through the infusion lumen 52 into one or both balloons and then to the tip 46 so that one or both balloons are automatically expanded when solution flows through the infusion lumen.

Alternatively, the method of use of the catheter can include the step of sequentially inflating/expanding the balloons or balloon portions via the same or separate inflation lumen as discussed above. The balloons can be inflated with a saline solution, the same solution being infused into the coronary sinus, or the like from separate sources. In this case, preferably the atrial balloon, or balloon portion, can be expanded first and then positioned into engagement with the ostium 22 in the right atrium 26. This locates the distal balloon, or balloon portion in the proper position within the coronary sinus. Thereafter, the distal balloon, or balloon portion, can be expanded into conformance with the inner walls of the coronary sinus 20 adjacent the ostium 22.

The catheter and method of its use according to the present invention can be used to enable global deliver of cardioplegia to enhance myocardial protection during open-heart operations that require cardiopulmonary bypass with retrograde perfusion. Myocardial protection of the right ventricle should particularly be improved, and right ventricular failure due to inadequate cardioplegia during prolonged cardiac operations should be capable of being avoided. The catheter and method also permits global delivery of intracardiac macromolecular complexes, pharmaceutical agents and gene therapies to the heart.

The catheter allows for maximal pressure gradient induction through maximal occlusion of the myocardial venous return system. The pressure gradient should facilitate and optimize delivery of pharmaceutical agents, gene therapy products, and other macromolecular complexes. The catheter should allow for maximum venous to interstitial pressure gradient for a given amount of flow and should prevent venous “shunting” or “steal” of drugs or gene therapy vectors delivered retrograde into veins draining into the more proximal portions of the coronary sinus. Eliminating “steal” or “shunt” pathways should result in proportionately more drug or vector gaining access to capillaries thereby facilitating diffusive and convective transport to tissues. Thus, an increase in delivery of macromolecular complexes, such as, proteins, DNA, or gene therapy vectors including adenovirus and adeno-associated virus should result. The expected increase in delivery at a given flow rate should be proportional to the increase in venous pressure at a given flow rate multiplied by the increase in tissue flow due to the elimination of the “shunt” fraction. For gene therapy vectors such as AAV, retrovirus, adenovirus, and others, the increase in interstitial delivery should directly correlate with an increase in myocyte transduction efficiency.

In one aspect, the invention provides a kit for use by a clinician or other personnel. Typically, such a kit will contain a catheter of the invention and, optionally, instructions for use thereof. In another embodiment, the kit will contain a macromolecular complex in a physiologically compatible saline solution and, optionally, instructions for dilution, and performing a method as described herein. The kit of the invention may also contain an oxygen-transporting agent and/or at least one disposable element of an extracorporeal circulatory support and oxygenation system.

A kit that is useful for performing the method of the invention is contemplated which comprises, in addition to the macromolecular complex and/or balloon catheter of the invention, at least one disposable element of an extracorporeal circulatory support and oxygenation system. Preferably, such a kit comprises all of the single-use components needed to perform the method of the invention, including a macromolecular complex, a vascular permeability-enhancing agent, a fluid delivery instrument such as a syringe or a length of peristaltic pump tubing, and a cannula such as a hollow bore needle adapted to fit a syringe. Such a kit may also contain a pharmaceutically acceptable carrier, a second cannula, an oxygen-transporting agent, a clearance solution which is substantially free of the macromolecular complex, one or more blood vessel occluding devices, such as a clamp, hemostat, or tourniquet, a disposable oxygenator, and the like.

While a preferred retrograde perfusion catheters and methods of its use have been described in detail, various modifications, alterations, and changes may be made without departing from the spirit and scope of the catheter and methods according to the present invention as defined in the appended claims.

1. A retrograde perfusion catheter (40, 40A, 40B), comprising:
a flexible, elongate cannula (42) having a distal end (44, 44A, 44B) of a size capable of insertion into a coronary sinus of a patient's heart;
an infusion lumen (52) extending longitudinally within said cannula (42) having an outlet (56) adjacent said distal end (44, 44A, 44B);
a first expandable balloon portion (66, 66A, 66B) attached to and surrounding a length of said distal end (44, 44A, 44B) of said cannula (42) upstream of said outlet (56) of said infusion lumen (52), said first balloon portion (66, 66A, 66B) being expandable to a size greater than the size of a coronary sinus ostium of a patient's heart; and
a second expandable balloon portion (68, 68A, 68B) attached to and surrounding a section of said distal end (44, 44A, 44B) of said cannula (42), adjacent said first balloon portion (66, 66A, 66B), at least a section of said second balloon portion (68, 68A, 68B) extending downstream of said first balloon portion (66, 66A, 66B) along said cannula (42) to a location closer to said outlet (56) of said infusion lumen (52), said second balloon portion (68, 68A, 68B) being inflatable from a size capable of introduction into the coronary sinus to an inflated size for sealing the coronary sinus;
whereby, positioning and inflation of said first and second balloon portions (66, 66A, 66B, 68, 68A, 68B) on opposite sides of the coronary sinus ostium enables the ostium and tissue of a right atrial chamber wall of a patient's heart to be captured therebetween to anchor the catheter (40, 40A, 40B) to the coronary sinus and to seal the coronary sinus at the ostium.

2. A retrograde perfusion catheter (40B) according to claim 1, wherein said first balloon portion (66B) and said second balloon portion (68B) form part of a single asymmetric balloon (70).

3. A retrograde perfusion catheter (40B) according to claim 2, wherein said balloon (70) has a substantially dumbbell or hourglass shape.

4. A retrograde perfusion catheter (40B) according to claim 2, further comprising an inflation lumen extending longitudinally within said cannula (42) in communication with only one of said first and second balloon portions (66B, 68B).

5. A retrograde perfusion catheter (40B) according to claim 4, wherein said balloon (70) has asymmetric and nonlinear capacitances or a threshold mechanism permitting said balloon portions (66B, 68B) to be sequentially expanded.

6. A retrograde perfusion catheter (40, 40A) according to claim 1, wherein said first balloon portion comprises a first balloon (66, 66A) and said second balloon portion comprises a separate second balloon (68, 68A).

7. A retrograde perfusion catheter (40) according to claim 6, wherein said first and second balloons (66, 68) are located side-by-side.

8. A retrograde perfusion catheter (40A) according to claim 6, wherein sections of said first and second balloons (66A, 68A) overlap.

9. A retrograde perfusion catheter (40, 40A, 40B) according to claim 1, wherein, when both said first and second balloon portions (66, 66A, 66B, 68, 68A, 68B) are inflated, said first balloon portion (66, 66A, 66B) has a greater diameter than said second balloon portion (68, 68A, 68B).

10. A retrograde perfusion catheter (40, 40A, 40B) according to claim 1, further comprising an inflation lumen extending longitudinally within said cannula (42) in communication with at least one of said first and second balloon portions (66, 66A, 66B, 68, 68A, 68B).

11. A retrograde perfusion catheter (40, 40A, 40B) according to claim 10, wherein said inflation lumen comprises a pair of separate inflation lumens, one being in communication with said first balloon portion (66, 66A, 66B) and the other being in communication with said second balloon portion (68, 68A, 68B).

12. A retrograde perfusion catheter (40, 40A, 40B) according to claim 1, wherein said infusion lumen (52) being in communication with at least one of said first and second balloon portions (66, 66A, 66B, 68, 68A, 68B) for automatic inflation thereof.

13. A retrograde perfusion catheter (40, 40A, 40B) according to claim 1, further comprising at least one additional lumen extending longitudinally within said cannula for pressure transduction or delivery of a second substance to the coronary sinus.

14. A retrograde perfusion catheter (40, 40A, 40B) according to claim 1, further comprising at least one additional lumen extending longitudinally within said cannula for monitoring fluid pressure within the coronary sinus.

15. A coronary sinus catheter (40, 40A, 40B) for the retrograde perfusion of cardioplegia solution in the coronary sinus of a patient's heart, comprising:
a flexible, elongate cannula (42) having a proximal end (48) and a distal end (44, 44A, 44B) of a size capable of insertion into the coronary sinus;
an infusion lumen (52) extending longitudinally within said cannula (42) and having an outlet (56) adjacent said distal end (44, 44A, 44B) and an inlet (54) adjacent said proximal end (48) for connection with a source of cardioplegia solution;
a first balloon portion (66, 66A, 66B) attached to and surrounding a length of said distal end (44, 44A, 44B) of said cannula (42) upstream of said infusion lumen outlet (56); and
a second balloon portion (68, 68A, 68B) attached to and surrounding a length of said distal end (44, 44A, 44B) of said cannula (42), said second balloon portion (68, 68A, 68B) extending closer to said infusion lumen outlet (56) than said first balloon portion (66, 66A, 66B);
said first balloon portion (66, 66A, 66B) being expandable to a greater diameter than said second balloon portion (68, 68A, 68B).

16. A retrograde perfusion catheter (40, 40A, 40B) according to claim 15, wherein said second balloon portion (68, 68A, 68B) is expandable from an uninflated diameter that is substantially equal to an outer diameter of said cannula (42) to an inflated diameter for engaging inner walls of a coronary sinus of a patient's heart.

17. A retrograde perfusion catheter (40, 40A, 40B) according to claim 16, wherein said first balloon portion (66, 66A, 66B) is expandable in a right atrium of a patient's heart to a size greater than a coronary sinus ostium of a patient's heart.

18. A retrograde perfusion catheter (40, 40A) according to claim 17, further comprising an inflation lumen extending longitudinally within said cannula (42) in communication with at least one of said first and second balloon portions (66, 66A, 66B, 68, 68A, 68B).

19. A retrograde perfusion catheter (40, 40A, 40B) according to claim 18, further comprising at least one additional lumen extending longitudinally within said cannula (42) for pressure transduction or delivery of a second substance to the coronary sinus.

20. A retrograde perfusion catheter (40, 40A, 40B) according to claim 15, further comprising a supply of cardioplegia solution connected to said infusion lumen (52) at said proximal end (48) of said cannula (42).

21. A method for retrograde coronary sinus perfusion of a patient's heart, comprising the steps of:
providing a catheter (40, 40A, 40B) having a proximal end (48) connected to a source of solution and a distal end (44, 44A, 44B) with expandable atrial and distal balloon portions (66, 66A, 66B, 68, 68A, 68B);
inserting at least a portion of the distal end (44, 44A, 44B) into the coronary sinus of the patient's heart;
expanding the atrial balloon portion (66, 66A, 66B) in the right atrium of the patient's heart and the distal balloon portion (68, 68A, 68B) within the coronary sinus to anchor the catheter (40, 40A, 40B) to the coronary sinus and to seal the coronary sinus adjacent its ostium; and
injecting the solution through the distal end (44, 44A, 44B) of the catheter (40, 40A, 40B) into the coronary sinus.

22. A method according to claim 21, wherein said atrial balloon portion (66, 66A, 66B) is expanded to a greater diameter than said distal balloon portion (68, 68A, 68B).

23. A method according to claim 22, wherein said atrial balloon portion (66, 66A, 66B) and said distal balloon portion (68, 68A, 68B) are expanded sequentially.

24. A method according to claim 22, wherein the atrial and distal balloon portions (66, 66A, 66B, 68, 68A, 68B) are located side-by-side and, after being expanded, sandwich the ostium therebetween.

25. A method according to claim 24, wherein, during said injection step, the solution flows directly into the right ventricle of the heart via the middle and small cardiac veins.

26. A method according to claim 21, wherein the solution is a cardioplegic solution.

27. A method according to claim 21, wherein the solution includes at least one of a macromolecular complex, a pharmaceutical agent, and a gene therapy vector.

28. A method for retrograde coronary sinus perfusion of a patient's heart, comprising the steps of:
providing a catheter (40, 40A, 40B) having a flexible, elongate cannula (42) with proximal and distal ends (48, 44, 44A, 44B), an infusion lumen (52) extending longitudinally within the cannula (42) with an outlet (56) adjacent the distal end (44, 44A, 44B), a first balloon portion (66, 66A, 6613) surrounding a length of the distal end (44, 44A, 44B) of the cannula (42) upstream of the outlet (56) of the infusion lumen (52), and a separate second balloon portion (68, 68A, 68B) surrounding a length of the distal end (44, 44A, 44B) of the cannula (42) adjacent the first balloon portion (66, 66A, 66B) at a location closer to the infusion lumen outlet (56);
inserting at least a portion of the distal end (44, 44A, 44B) of the cannula (42) into a coronary sinus of a patient's heart so that the second balloon portion (68, 68A, 68B) is at least partially positioned within the coronary sinus and the first balloon portion (66, 66A, 66B) is positioned in the right atrium of the heart adjacent and exterior the coronary sinus; and
expanding the first and second balloon portions (66, 66A, 66B, 68, 68A, 68B) to capture tissue of the right atrial chamber wall and the coronary sinus ostium between the first and second balloon portions (66, 66A, 66B, 68, 68A, 68B) to anchor the catheter (40, 40A, 40B) to the coronary sinus and to seal the ostium of the coronary sinus;
injecting a solution into the infusion lumen (52) so that it flows through the outlet (56) of the cannula (42) into the coronary sinus.

29. A method according to claim 28, wherein said first balloon portion (66, 66A, 66B) is expanded to a larger diameter than said second balloon portion (68, 68A, 68B).

30. A method according to claim 29, wherein said first and second balloon portions (66, 66A, 66B, 68, 68A, 68B) are expanded in sequence.

31. A method according to claim 30, wherein said first balloon portion (66, 66A, 66B) is expanded in the right atrium against the ostium, and thereafter, the second balloon portion (68, 68A, 68B) is expanded within the coronary sinus.

32. A method according to claim 28, wherein the solution is a cardioplegic solution.

33. A method according to claim 28, wherein the solution includes at least one of a macromolecular complex, a pharmaceutical agent, and a gene therapy vector.

34. A method according to claim 28, wherein said second balloon portion (68, 68A, 68B) is inflated at a location within the coronary sinus upstream of a junction of the middle and small cardiac veins with the coronary sinus to permit the solution to flow directly into the right ventricle of the heart via the middle and small cardiac veins.

35. Use of a catheter (40, 40A, 40B) having a proximal end (48) connected to a source of solution and a distal end (44, 44A, 44B) with expandable atrial and distal balloon portions (66, 66A, 66B, 68, 68A, 68B) to perform retrograde coronary sinus perfusion of a patient's heart, at least a portion of the distal end (44, 44A, 44B) is inserted into the coronary sinus of the patient's heart, and the atrial balloon portion (66, 66A, 66B) is expanded in the right atrium of the patient's heart and the distal balloon portion (68, 68A, 68B) is expanded within the coronary sinus to anchor the catheter (40, 40A, 40B) to the coronary sinus and to seal the coronary sinus adjacent its ostium, thereafter the solution is injected through the distal end (44, 44A, 44B) of the catheter (40, 40A, 40B) into the coronary sinus.
